(19) United States
(12) Patent Application Publication
(10) Pub. No.: US 2013/0022645 A1
(43) Pub. Date: Jan. 24, 2013

(54) PHARMACEUTICAL FORMULATIONS OF BISPHOSPHONATE WITH ENHANCED ORAL BIOAVAILABILITY

(71) Applicants: Mi-Won SON, Yongin-si (KR); Sun-Woo JANG, Seoul (KR); Jeong-Soo KIM, Yongin-si (KR); Dong-Sung RYU, Yongin-si (KR); Wan-Sung KU, Yongin-si (KR)

(72) Inventors: Mi-Won SON, Yongin-si (KR); Sun-Woo JANG, Seoul (KR); Jeong-Soo KIM, Yongin-si (KR); Dong-Sung RYU, Yongin-si (KR); Wan-Sung KU, Yongin-si (KR)

(73) Assignee: DONG-A PHARM. CO., LTD., Seoul (KR)

(21) Appl. No.: 13/276,873

(22) Filed: Oct. 19, 2011

(30) Foreign Application Priority Data
Jul. 22, 2011 (KR) 10-2011-0073130

(51) Int. Cl. A61K 9/00; A61K 31/66

(52) U.S. Cl. 424/400; 514/108

(57) ABSTRACT

Disclosed is an oral bisphosphonate formulation characterized by an enhanced clinical bioavailability of bisphosphonate and by the use of phytic acid and a delayed release means for releasing bisphosphonate at a site of the lower gastrointestinal tract. Having a low phytic acid content, the oral bisphosphonate formulation guarantees high safety to the patient. Moreover, the oral formulation is designed to allow the patients to take the medicament, together with food intake, at a bioavailability as high as that of an empty stomach, thus improving the convenience of drug administration for the patient. Therefore, the oral formulation is expected to provide higher therapeutic effects for osteoporosis.

### BACKGROUND OF THE INVENTION

1. Field of the Invention

The present invention relates to an oral bisphosphonate-based drug with enhanced bioavailability for the treatment of osteoporosis.

2. Description of the Related Art

Functioning to prevent bone resorption without negatively affecting the formation and mineralization of bones, bisphosphonate-based drugs are used to treat diseases characterized by abnormal bone absorption such as osteoporosis, Paget's disease and the like.

Among the bisphosphonate-based drugs in such medical uses are alendronate, etidronate, pamidronate, risedronate, tiludronate, ibandronate, zoledronate, clodronate, incadronate, minodronate neridronate, olpadronate, and cimadronate. Of them, alendronate, risedronate, ibandronate, and zoledronate are widely used because they can elicit therapeutically useful effects even in small doses thanks to their potent pharmaceutical activity.

However, the oral bioavailability of bisphosphonates in humans is known to lie below 1% [J. Lyn, Bone, 18: 75-85 (1996), Ezra et al., Adv. Drug Del. Rev. 42: 175-195 (2000)], and absorption is greatly reduced when administered with food and drinks, especially in the presence of minerals. Therefore bisphosphonates should never be given at mealtime and never together with mineral ions-containing diets. Otherwise, the bioavailability of phisphosphonates are greatly reduced. The nearer the time interval between intake of foods and bisphosphonates is, the greater the reduction of bioavailability is. For example, it was found that taking bisphosphonates 30 min after a meal reduced bioavailability by as high as 50% relative to 4 hours after a meal [Mitchell et al., Pharm. Res. 14: 5609 (1996), Mitchell et al., Br. J. Clin. Pharmacol. 48: 536-542 (1999)]. This trait is reported to be attributed to mineral ions present in the gastrointestinal tract [Mahet al., Am. J. Clin. Nutr. 56: 410-416 (1992)].

Originally, bisphosphonate compounds were developed as chelates to soften hard water and thus exhibit high affinity for mineral ions such as calcium and magnesium. When encountering these ions, bisphosphonate compounds form insoluble complexes which lead to the absorption of bisphosphonates. Due to this property, the practical dosing recommendation of bisphosphonate formulations is that patients should take the drug at least 30 min before meals. For example, as for ibandronate formulations, they should be dosed 60 min before a meal.

There has been therefore a need for pharmaceutical formulations comprising bisphosphonate that allow the patients to take the medicament, together with food intake, at a bioavailability as high as that on an empty stomach. For this, the following attempts have been made to overcome the low oral bioavailability of bisphosphonates.

U.S. Pat. No. 4,980,171 discloses oral pharmaceutical composition comprising a bisphosphonic acid derivative and sodium laurylsulfate which are improved in bioavailability. Korean Patent Laid-Open Publication No. 2009-0037851 discloses a pharmaceutical composition comprising a bisphosphonate in combination with a medium chain fatty acid or a salt thereof as an absorption enhancer. In International Patent Publication No. WO 98/14196, a combination of citric acid and tartaric acid is used to enhance the absorption of bisphosphonate. International Patent Publication No. WO 00/061111 introduces various surfactants such as poloxamer, sodiumlaurylsulfate, etc. as an absorption enhancer of bisphosphonate.

These absorption enhancers are known to increase the bioavailability of bisphosphonates by inhibiting the formation of insoluble complexes with mineral ions or reversibly or irreversibly controlling the permeability of bisphosphonates into intestinal mucosal membranes.

In these attempts, however, excessive amounts of enhancers are required to attain desired absorption of bisphosphonates. The amounts of the enhancers used are too high for a dose of the medicament so that they are impossible to apply to practical oral formulations [Janner et al., Calcif. Tissue Int. 49: 280-283 (1991)]. Further, although oral formulations with a high content of enhancers are made to enhance the bioavailability of bisphosphonates, excessive amounts of the enhancer, when administered, may irritate the intestinal mucosa, incurring a problem in the safe use of the medicament. In vivo assays that have examined the effects on intestinal mucosa in rabbits showed that various absorption enhancers such as sodium laurylsulfate, EDTA, etc., irritate the intestinal mucosa to cause, for example, the delamination of the intestinal mucosa and the edema of submucous layers [M. Yonezawa, Nihon Univ. J. Med. 19: 125-141 (1977)].

In addition, after intake of a diet containing minerals, the minerals are better absorbed in the digestive tract which is farther away from the intestine, and the minerals are present at low concentration in the intestinal tract [Mahet al., Am. J. Clin. Nutr. 56: 410-416 (1992)]. However, it has already been known that the oral bioavailability of bisphosphonates cannot be enhanced only by a drug delivery system targeting the intestine, such as enteric coating of tablets [Mitchell et al., Pharm. Res. 14: 5609 (1996)]. Accordingly, in order to minimize the inhibition that diet minerals have on the absorption of bisphosphonate and to elicit the desired increase in the bioavailability of bisphosphonate, it is needed to design the release of bisphosphonate and an absorption enhancer at a specific site of the intestine which has as low a mineral concentration as does an empty stomach.

J. Lyn reported that a chelating agent such as EDTA can increase the bioavailability of bisphosphonate [J. Lyn, Bone, 18: 75-85 (1996)], and U.S. Pat. Nos. 7,645,459 and 7,645,460 also describe an in vitro assay to demonstrate the ability of a chelating agent to enhance the absorption of bisphosphonate.

Paying attention to the article of J. Lyn [J. Lyn, Bone, 18: 75-85 (1996)], the present inventors have studied a strategy for enhancing the oral bioavailability of bisphosphonate formulations containing a chelating agent.

Results from in vitro tests conducted by the present inventors indicated that phytic acid, serving as a chelating agent, cannot show the same inhibitory activity against calcium-risedronate complex formation as is obtained by EDTA without using phytic acid in an amount twice that of EDTA. Because too much phytic acid is required to substantially enhance the bioavailability of bisphosphonates, the present inventors finally regarded the use of phytic acid as being clinically impossible.

Surprisingly, however, animal tests with enteric coating drug delivery systems of bisphosphonate formulations containing phytic acid or EDTA gave the reverse finding, overturning the prediction of those skilled in the pharmaceutical field who naturally deduced from the in vitro tests about the inhibition against calcium-risedronate complex formation, that bisphosphonate formulations containing phytic acid increase in both AUC and Cmax three times greater than do those containing EDTA so that phytic acid can enhance the oral bioavailability of bisphosphonates to a clinically practicable degree, culminating in the present invention.

### SUMMARY OF THE INVENTION

It is therefore an object of the present invention to provide an oral bisphosphonate formulation the bioavailability of which is sufficiently enhanced and so is clinically useful.

### BREIF DESCRIPTION OF THE DRAWINGS

The above and other objects, features and other advantages of the present invention will be more clearly understood from the following detailed description taken in conjunction with the accompanying drawings, in which:

FIG. 1 is a graph showing the in vitro inhibitory activity of phytic acid and EDTA against the complexation of bisphosphonate with calcium in 50 mL solutions containing 50 mg of calcium;

FIG. 2 is a graph showing the in vitro inhibitory activity of phytic acid and EDTA against the complexation of bisphosphonate with calcium in 50 mL solutions containing 10 mg of calcium;

FIG. 3 is a graph showing the in vitro inhibitory activity of phytic acid and EDTA against the complexation of bisphosphonate with calcium in 50 mL solutions containing 5 mg of calcium, with the plots of Reference Examples 12 to 15 enlarged in FIG. 3a;

FIG. 4 is a graph showing the data of in vitro release assays with the enteric coating formulation of Example 1, the formulation lacking a chelating agent of Comparative Example 1, and the enteric coating formulation containing EDTA as a chelating agent of Comparative Example 2; and

FIG. 5 is a graph showing the data of in vivo assays for bioavailability of the enteric coating formulation of Example 1, the formulation lacking a chelating agent of Comparative Example 1, and the enteric coating formulation containing EDTA as a chelating agent of Comparative Example 2.

### DESCRIPTION OF THE PREFERRED EMBODIMENTS

In accordance with an aspect thereof, the present invention addresses an oral formulation comprising a bisphosphonate drug and phytic acid in combination with a release delaying means for targeting the release of bisphosphonate and phytic acid to the lower gastrointestinal tract.

The bisphosphonate drug the bioavailability of which can be enhanced sufficiently for clinical use in accordance with the present invention may be selected from among alendronate, risedronate, ibandronate, zoledronate, pamidronate, clodronate, tiludronate, cimadronate and minodronate. Preferable are alendronate, risedronate, and ibandronate, risedronate being more preferred.

In addition, in an oral formulation comprising a release delaying means for targeting the release of bisphosphonate to the lower gastrointestinal tract, the weight ratio of bisphosphonate to phytic acid may range from 1:0.5 to 1:3, depending on the desired increase of bioavailability.

Phytic acid (inositol hexakisphosphate) is the principal storage form of phosphorus in many plant tissues, especially bran and seeds, and is called phytate when in salt form. Phytic acid or phytate is not digestible by humans, however, so it is not a source of either inositol or phosphate if eaten directly. Morever, it is known to serve as a chelating agent which can form complexes with various minerals such as iodine (I), zinc (Zn), calcium (Ca), magnesium (Mg), cadmium (Cd), etc.

U.S. Pat. Nos. 7,645,459 and 7,645,460 describe that the ability of a chelating agent to enhance the absorption of bisphosphonate can be predicted from the complexation stability constant K of the chelating agent. Log K is defined as a ratio between amounts of metal ions consumed for complexation and free metal ions. The practical significance of formation constants is that a high log K value means a large ratio of chelated to unchelated metal ions, that is, a high ability to form complexes.

Also, the prior art discloses that in order to enhance the bioavailability of bisphosphonate sufficiently, the chelating agent must have a log K which is at least 4 units higher than the bisphosphonate-metal ion complex. In order to facilitate the absorption of bisphosphonate, a chelating agent having a lower log K must be added in highly excessive amounts over the bisphosphonate, but this is impossible to clinically embody in practice.

Moreover, the mechanism in which a chelating agent facilitates the absorption of bisphosphonate may be explained as the degree to which the chelating inhibits the activity of bisphosphonate to react with minerals (calcium, magnesium, etc.) in the gastrointestinal tract to form insoluble complexes. Thus, for example, after bisphosphonate and a chelating agent are added simultaneously to a solution containing a predetermined amount of calcium ions, the concentration of bisphosphonate in the solution may be plotted against time to understand the inhibitory activity of the chelating agent against complexation with calcium ions.

Conventional chelating agents (EDTA, etc.) are almost impossible to apply in clinical practice because a large amount of them is required to enhance the bioavailability of bisphosphonate. Results from in vitro assays indicate that phytic acid shows the same inhibitory activity against complexation as does EDTA only when it is used in an amount at least twice the amount of EDTA.

Hence, even those skilled in the pharmaceutical field would determine that it is impossible to apply phytic acid to bisphosphonate formulations because it should be used in an excessive amount to confer clinically useful bioavailability on them.

On the basis of the finding, however, that the concentration of bisphosphonate tends not to be reduced in the presence of phytic acid compared to when it exists alone, although phytic acid does not exhibit inhibitory activity against complexation in vitro without using it in an excessive amount twice as much as the amount of EDTA, the present inventors carried out animal tests with phytic acid.

Surprisingly, overturning the prediction which those skilled in the pharmaceutical field can naturally deduce from the in vitro tests for inhibition against calcium-risedronate complex formation, the animal tests with enteric coating drug delivery systems targeting bisphosphonate formulations containing phytic acid or EDTA or an immediate release formulation to a specific site of the small intestine showed that bisphosphonate formulations containing phytic acid increased in both AUC and Cmax three to five times greater than do those containing EDTA so that phytic acid guaranteed bisphosphonates that have superior oral bioavailability.

Bisphosphonate-based drugs are known to be absorbed in the gastrointestinal tract [Mitchell et al, Pharm. Res. 15: 228-232 (1998)] and it is reported that it is impossible to enhance the bioavailability of bisphosphonate by means of an intestine-targeting drug delivery system, such as enteric coating capsule [Mitchell et al., Pharm. Res. 14: S609 (1996)]. Thus, the enhancement of the bioavailability of bisphosphonate in the animal tests is entirely attributed to phytic acid.

Therefore, in accordance with another aspect thereof, the present invention provides an enteric coating formulation as a drug delivery system allowing the release of phytic acid and bisphosphonate to the small intestine so as to maximize the phytic acid-induced enhancement of bioavailability of bisphosphonate. Unless the release of phytic acid and bisphosphonate targets the small intestine, the inhibitory activity of phytic acid against the formation of insoluble mineral-bisphosphonate complexes is decreased due to the presence of abundant mineral ions in the upper gastrointestinal tract, which results in a decrease in the bioavailability of bisphosphonate.

Non-limiting examples of the delayed release means targeting phytic acid and bisphosphonate to a specific site of the small intestine include pH-dependent enteric coating drug delivery systems and time-dependent delayed release systems.

According to the present invention, an enteric coating drug delivery system that allows a drug to be released in a pH-dependent manner is used to formulate bisphosphonate and phytic acid into an oral dosage form which can slowly release the medicament at a site of the lower gastrointestinal tract, that is, one selected from among the duodenum, the jejunum, the ileum and the ascending colon, during passage therethrough.

Alternatively, a delayed release drug delivery system that allows drug release in a time-dependent manner is used to formulate bisphosphonate and phytic acid into an oral dosage form which can release the bisphosphonate and the phytic acid within 30 min at a site of the lower gastrointestinal tract, that is, one selected from among the duodenum, the jejunum, the ileum and the ascending colon, during passage therethrough. Further, the oral dosage form may be designed to release the medicament at different sites of the lower gastrointestinal tract.

The oral formulation comprising a bisphosphonate drug and phytic acid in combination with a release delaying means for targeting the release of bisphosphonate and phytic acid to the lower gastrointestinal tract in accordance with the present invention may be administered at regular time intervals selected from among once a day, once a week, once a month, twice a month and three times a month, depending on the dose of the bisphosphonate, and preferably administered once a week.

Provided with a delayed release drug delivery system for targeting the release of phytic acid and bisphosphonate, the oral formulation of the present invention is enhanced in the bioavailability of bisphosphonate to allow the clinical application of bisphosphonate.

In addition, the oral formulation of the present invention was found to guarantee highly sufficient bioavailability to bisphosphonate, compared to conventional chelating agents, as measured by animal tests in which the oral formulation comprising phytic acid increased the bioavailability of bisphosphonate 3-5-fold in terms of Cmax and AUC, relative to immediate release formulations or enteric coating formulation containing EDTA. Thus, even a small amount of phytic acid promises bisphosphonate sufficient bioavailability, so being unlikely to irritate the intestinal mucosa, with the guaranty of high safety to the patient. Moreover, the oral formulation of the present invention is designed to allow the patients to take the medicament, together with food intake, at a bioavailability as high as that of an empty stomach, thus improving the convenience of drug administration for a patient. Consequently, the oral formulation of the present invention is expected to provide higher therapeutic effects on osteoporosis, compared to conventional ones.

A better understanding of the present invention may be obtained through the following examples which are set forth to illustrate, but are not to be construed as limiting the present invention.

Herein, in vitro tests in which after bisphosphonate and a chelating agent were simultaneously added to a solution containing a predetermined amount of calcium ions, correlation between time and residual bisphosphonate in the solution was determined were performed as explained in the following Reference Examples.

### REFERENCE EXAMPLES 1 TO 15

### Preparation of Solutions Containing Bisphosphonate Plus EDTA or Phytic Acid

Various 50 mL solutions containing 50 mg, 10 mg and 5 mg of calcium in combination with 50 mg and 100 mg of EDTA, or 100 mg, 200 mg and 35 mg of risedronate as a chelating agent were prepared and used respectively in Reference Examples 1 to 15.

The compositions and contents of ingredients in the solutions of Reference Examples 1 to 15 are summarized in Table 1, below.

TABLE 1

| Ref. | 50 ml | | | |
|---|---|---|---|---|
| Ex | Ca (mg) | EDTA (mg) | Phytic Acid (mg) | Risedronate (mg) |
| 1 | 50 | — | — | 35 |
| 2 | 50 | 50 | — | 35 |
| 3 | 50 | 100 | — | 35 |
| 4 | 50 | — | 100 | 35 |
| 5 | 50 | — | 200 | 35 |
| 6 | 10 | — | — | 35 |
| 7 | 10 | 50 | — | 35 |
| 8 | 10 | 100 | — | 35 |
| 9 | 10 | — | 100 | 35 |
| 10 | 10 | — | 200 | 35 |
| 11 | 5 | — | — | 35 |
| 12 | 5 | 50 | — | 35 |
| 13 | 5 | 100 | — | 35 |
| 14 | 5 | — | 100 | 35 |
| 15 | 5 | — | 200 | 35 |

### REFERENCE TEST EXAMPLE 1

### Comparison of In vitro Inhibitory Activity Against Insoluble Bisphosphonate Complex Formation Between Phytic Acid and EDTA

The mechanism in which a chelating agent facilitates the absorption of bisphosphonate can be explained as the degree to which the chelating inhibits bisphosphonate's activity of reacting with minerals (calcium, magnesium, etc.) in the gastrointestinal tract to form insoluble complexes. Thus, in vitro tests for assaying the inhibitory activity of phytic acid or EDTA against the complexation of bisphosphonate in calcium-containing solutions were conducted in Reference Test Example 1.

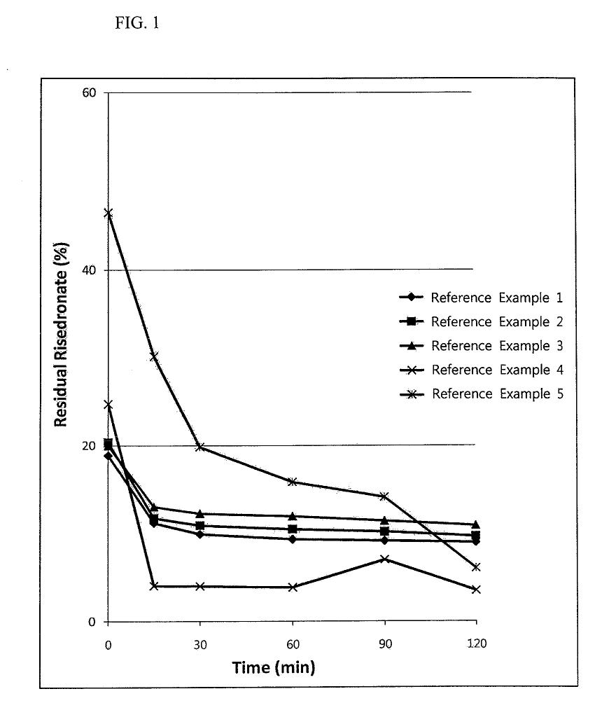

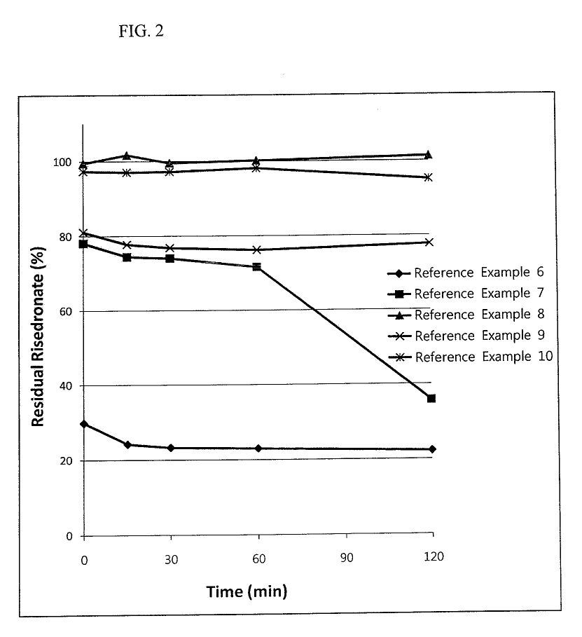

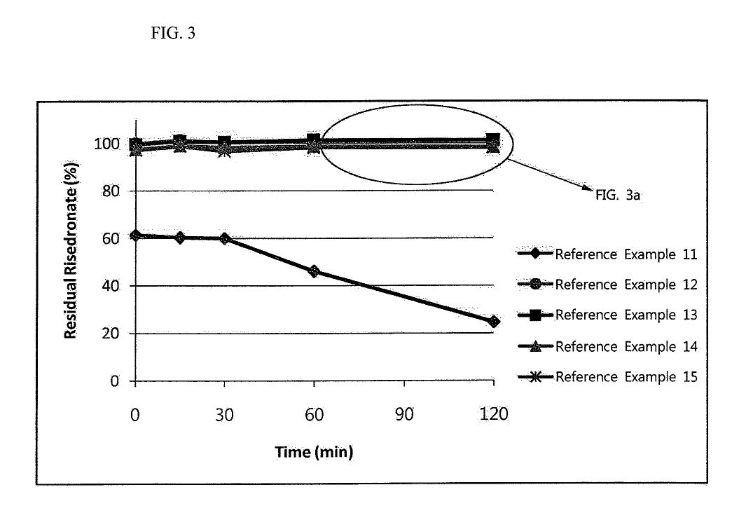

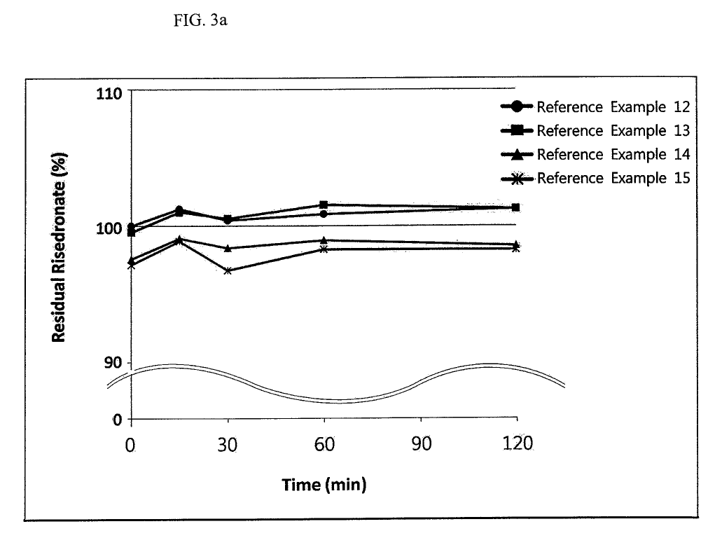

In a container with a cap, 25 mL of each of the solutions prepared in Reference Example 1 to 15 was placed, followed by agitation at 100 rpm for 120 min in an agitation bath maintained at 37° C. Approximately 1 mL of each of the solutions was taken at time points (immediately after preparation, and 15, 30, 60, 90 and 120 min after agitation) and allowed to pass through 0.45 μm PVDF membrane filters to remove precipitates. Residual resedronate in the solutions was quantitatively analyzed by HPLC. The results are summarized in Tables 2 to 4 and shown in FIGS. 1 to 3.

TABLE 2

| | % | 0 | 15 | 30 | 60 | 90 | 120 | 180 |
|---|---|---|---|---|---|---|---|---|
| Ref. | Risedronate | 18.9 | 11.2 | 9.9 | 9.3 | 9.1 | 8.9 | 8.3 |
| Ex. 1 | | | | | | | | |
| Ref. | Risedronate | 20.4 | 11.7 | 10.9 | 10.5 | 10.2 | 9.7 | 9.0 |
| Ex. 2 | EDTA 50 | | | | | | | |
| Ref. | Risedronate | 20.0 | 13.0 | 12.2 | 11.9 | 11.4 | 10.9 | 10.4 |
| Ex. 3 | EDTA 100 | | | | | | | |
| | Risedronate | 18.8 | 13.0 | 7.5 | 7.4 | 7.3 | 7.0 | 7.0 |
| | Phytic acid 20 | | | | | | | |
| | Risedronate | 10.4 | 5.1 | 5.0 | 4.9 | 4.9 | 4.6 | 4.5 |
| | Phytic acid 50 | | | | | | | |
| Ref. | Risedronate | 24.8 | 4.0 | 4.0 | 3.8 | 7.0 | 3.5 | 3.2 |
| Ex. 4 | Phytic acid 100 | | | | | | | |
| Ref. | Risedronate | 46.5 | 30.2 | 19.8 | 15.8 | 14.1 | 6.0 | 13.6 |
| Ex. 5 | Phytic acid 200 | | | | | | | |

TABLE 3

| | % | 0 | 15 | 30 | 60 | 120 |
|---|---|---|---|---|---|---|
| Ref. | Risedronate | 30 | 24 | 23 | 23 | 22 |
| Ex. 6 | | | | | | |
| Ref. | Risedronate | 78 | 74 | 74 | 72 | 36 |
| Ex. 7 | EDTA 50 | | | | | |
| Ref. | Risedronate | 99 | 102 | 99 | 100 | 101 |
| Ex. 8 | EDTA 100 | | | | | |
| | Risedronate | 53 | 47 | 47 | 47 | 47 |
| | Phytic acid 20 | | | | | |
| | Risedronate | 70 | 64 | 63 | 65 | 63 |
| | Phytic acid 50 | | | | | |
| Ref. | Risedronate | 81 | 78 | 77 | 76 | 78 |
| Ex. 9 | Phytic acid 100 | | | | | |
| Ref. | Risedronate | 97 | 97 | 97 | 98 | 95 |
| Ex. 10 | Phytic acid 200 | | | | | |

TABLE 4

| | % | 0 | 15 | 30 | 60 | 120 |
|---|---|---|---|---|---|---|
| Ref. | Risedronate | 61 | 60 | 60 | 46 | 25 |
| Ex. 11 | | | | | | |
| Ref. | Risedronate | 100 | 101 | 100 | 101 | 101 |
| Ex. 12 | EDTA 50 | | | | | |
| Ref. | Risedronate | 100 | 101 | 101 | 102 | 101 |
| Ex. 13 | EDTA 100 | | | | | |
| | Risedronate | 83 | 83 | 82 | 82 | 77 |
| | Phytic acid 20 | | | | | |
| | Risedronate | 91 | 92 | 92 | 92 | 92 |
| | Phytic acid 50 | | | | | |
| Ref. | Risedronate | 98 | 99 | 98 | 99 | 99 |
| Ex. 14 | Phytic acid 100 | | | | | |
| Ref. | Risedronate | 97 | 99 | 97 | 98 | 98 |
| Ex. 15 | Phytic acid 200 | | | | | |

As is apparent from the data of Tables 2 to 4 and FIGS. 1 to 3, both phytic acid and EDTA tend to inhibit calcium-risedronate complexation in a dose-dependent manner. Accordingly, as reported by J. Lyn [J. Lyn, Bone, 18: 75-85 (1996)1, both phytic acid and EDTA would be expected to increase the oral bioavailability of bisphosphonate thanks to their inhibitory activity against calcium-risedronate complexation.

Meanwhile, data of Reference Examples 6 to 10 showed that phytic acid must be used in an amount twice as large as that of EDTA so as to inhibit calcium-risedronate complexation to the same degree as in EDTA. Phytic acid was observed to be poorer in inhibitory activity against complexation than was EDTA, as measured by in vitro tests.

Further, a bisphosphonate product (brand name: Atelvia®), approved by the FDA in October 2010, which contains a high content of EDTA to enhance the bioavailability of bisphosphonate, was reported to cause abdominal pain with significance, compared to bisphosphonate alone, when administered on an empty stomach, in clinical trial phase III [USFDA, Atelvia Biopharmaceutics Clinical Pharmacokinetics review]. Hence, excessive amounts of phytic acid are required to confer useful bioavailability to bisphosphonate formulations, which makes it impossible to apply phytic acid to clinical formulations, according to the viewpoint furnished by the in vitro tests.

Surprisingly, overturning the prediction which those skilled in the pharmaceutical field who could naturally deduce from the in vitro tests for the inhibition against calcium-risedronate complexation, the present inventors newly found that phytic acid can guarantee higher bioavailability to bisphosphonate than can EDTA.

Below, a detailed description will be given of oral formulations provided with a means for targeting the release of bisphosphonate, phytic acid or a combination thereof to the small intestine, with reference to the Examples and Test Examples.

### EXAMPLE 1

### Preparation of Enteric Coating Tablet Comprising Phytic Acid and Risedronate

An enteric coating tablet comprising phytic acid and risedronate was prepared to have the composition of Table 5.

Risedronate sodium, colloidal silica, phytic acid, sodium starch glycolate and crospovidone were combined into a phytic acid solution which was then granulated and dried. The granules thus obtained were sized through an 18-mesh sieve, mixed with magnesium stearate for 3 min and tabletted into a nuclear tablet which was then coated with an aqueous suspension of Acryleze to afford an enteric coating tablet.

TABLE 5

| Ingredient | Content (mg) |
|---|---|
| Risedronate sodium | 35.00 |
| Colloidal silica | 90.00 |
| Phytic acid | 90.00 |
| Sodium starch glycolate | 25.00 |
| Crospovidone | 10.00 |
| Mg stearate | 1.25 |
| Acryl EZE | 25.13 |
| Total | 276.38 |

### EXAMPLE 2

### Preparation of Enteric Coating Tablet Comprising Phytic Acid and Alendronate

An enteric coating tablet comprising phytic acid and alendronate was prepared to have the composition of Table 6.

Alendronate sodium, colloidal silica, phytic acid, sodium starch glycolate and crospovidone were combined into a phytic acid solution which was then granulated and dried. The granules thus obtained were sized through a 18-mesh sieve, mixed with magnesium stearate for 3 min and tabletted into a nuclear tablet which was then coated with an aqueous suspension of Acryleze to afford an enteric coating tablet.

TABLE 6

| Ingredient | Content (mg) |
|---|---|
| Alendronate sodium | 70.00 |
| Colloidal silica | 90.00 |
| Phytic acid | 90.00 |
| Sodium starch glycolate | 25.00 |
| Crospovidone | 10.00 |
| Mg stearate | 1.43 |
| Acryl EZE | 28.64 |
| Total | 315.07 |

### EXAMPLE 3

### Preparation of Enteric Coating Tablet Comprising Phytic Acid and Ibandronate

An enteric coating tablet comprising phytic acid and ibandronate was prepared to have the composition of Table 7.

Ibandronate sodium, colloidal silica, phytic acid, sodium starch glycolate and crospovidone were combined into a phytic acid solution which was then granulated and dried. The granules thus obtained were sized through an 18-mesh sieve, mixed with magnesium stearate for 3 min and tabletted into a nuclear tablet which was then coated with an aqueous suspension of Acryleze to afford an enteric coating tablet.

TABLE 7

| Ingredient | Content (mg) |
|---|---|
| Ibandronate sodium | 150.00 |
| Colloidal silica | 90.00 |
| Phytic acid | 90.00 |
| Sodium starch glycolate | 25.00 |
| Crospovidone | 10.00 |
| Mg stearate | 1.83 |
| Acryl EZE | 36.68 |
| Total | 403.51 |

### COMPARATIVE EXAMPLE 1

### Commercial Risendronate Product without Chelating Agent

A commercially available product containing 35 mg of risedronate without an absorption enhancer (Actonel®, produced by P&G Pharmaceuticals Inc., manufactured by OSG Norwich Pharmaceuticals Inc., marketed by Sanofi-Aventis Korea, Lot No. C009) was purchased.

### COMPARATIVE EXAMPLE 2

### Preparation of Enteric Coating Tablet Comprising EDTA and Risedronate

Risedronate sodium, edetate disodium dihydrate, silicate microcrystalline cellulose and sodium starch glycolate were weighed and mixed together as shown in the composition of Table 8. The mixture was further combined with stearic acid and magnesium stearate for 3 min, and tabletted into a nuclear tablet which was then coated with an aqueous suspension of Acryleze to afford an enteric tablet containing EDTA and risedronate, like the EDTA-containing bisphosphonate product (brand name: Atelvia®), approved by the FDA.

TABLE 8

| Ingredient | Content (mg) |
|---|---|
| Risedronate sodium | 35.00 |
| Edentate disodium dihydrate | 100.00 |
| Silicate microcrystalline cellulose | 85.80 |
| Sodium starch glycolate | 6.00 |
| Stearic acid | 12.00 |
| Mg stearate | 1.20 |
| Acryl EZE | 24.00 |
| Total | 264.00 |

### TEST EXAMPLE 1

### In vitro Assay for Release

An in vitro assay was performed according to the Delayed-release dosage form dissolution method A (paddle method, 50 rpm) of the release test section of the U. S. pharmacopoeia. Requirements for the release assay are summarized in Table 9, below.

TABLE 9
Conditions of Release Assay

| | |
|---|---|
| Solution | 1^{st} phase: 0.1N HCI 750 ml (2 hrs) |
| | 2^{nd} phase: 250 ml of 0.2M tribasic sodium |
| | phosphate was added to adjust pH to 6.8 after |
| | the 1^{st} phase (1 hr) |
| Instrument | Dissociation tester, paddle method |
| Agitation Speed | 50 rpm |
| Agitation Temp. | 37 ± 0.5° C. |

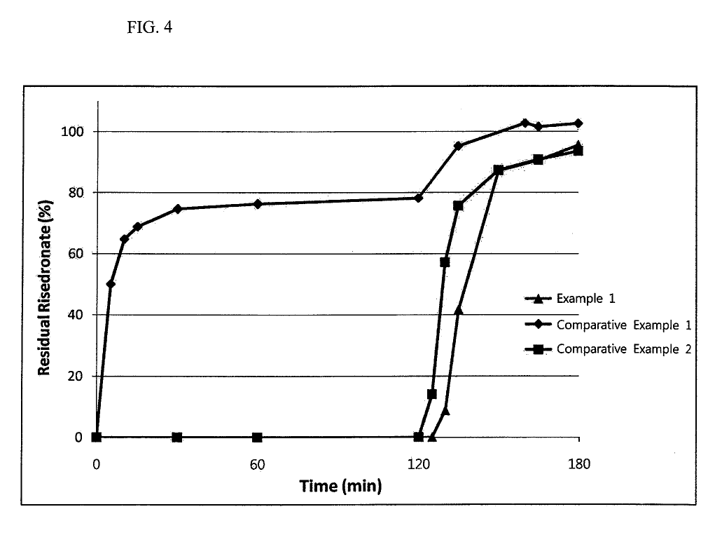

FIG. 4 is a graphic representation of the results from the in vitro release assay of formulations of Example 1 and Comparative Examples 1 and 2.

As shown in FIG. 4, the immediate release formulation of Comparative Example 1 reached a release rate of 80% 30 min after being subjected to the acidic condition and released most risedronate in the second phase. On the other hand, no release occurred in the formulations of Example 1 and Comparative Example 2 in the first phase of the acidic condition due to their enteric coat while more than 85% of the risedronate was released within 30 min after being subjected to the second phase of the alkaline condition. Thus, the formulations of Example 1 and Comparative Example 2 were regarded as enteric coating tablets having a perfect coat.

### TEST EXAMPLE 2

### In Vivo Assay of Bisphosphonate for Bioavailability in Beagle Dog

Bisphosphonate formulations comprising phytic acid or EDTA of Example 1 and Comparative Examples 1 and 2 were administered to beagle dogs and compared for bioavailability

Evaluation for bioavailability was conducted in 20 male beagle dogs, each weighing 10-15 kg. In this context, open, randomized, parallel-group study was performed. For use in the study, animals were starved, except for water, overnight before the day of administration and fed 4 hours before drug administration. Irrespective of weight difference, each animal was administered with 35 mg of risedronate sodium.

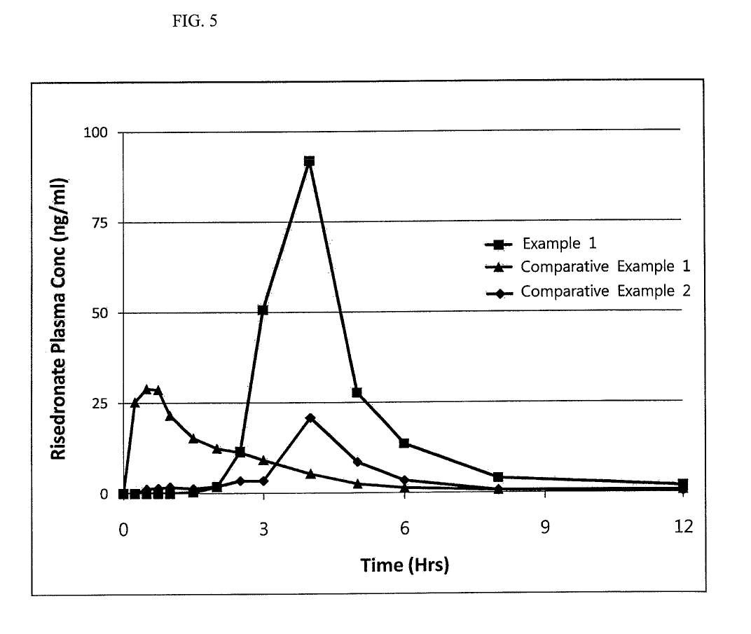

Blood samples were taken from the external jugular vein at predetermined time points: immediately before administration, 0.25, 0.5, 0.75, 1, 1.5, 2, 2.5, 3, 4, 5, 6, 8, 12 and 24 hours after administration. Approximately 5 mL of blood was sampled, placed in heparinized tubes, and centrifuged to separate plasma, followed by storage at −80° C. before analysis. After SPE treatment, serum drug levels were determined using LC/MS/MS, and expressed as residual plasma concentration (ng/ml) of risedronate over time in Table 10 and FIG. 5.

TABLE 10
Assay for Bioavailability

| | C. Ex. 1 | C. Ex. 2 | Ex. 1 |
|---|---|---|---|
| Time (hr) | Avg. (n = 10) | Avg. (n = 5) | Avg. (n = 5) |
| 0 | 0.0 | 0.0 | 0.0 |
| 0.25 | 25.2 | 0.0 | 0.0 |
| 0.5 | 28.8 | 1.2 | 0.0 |
| 0.75 | 28.6 | 1.5 | 0.0 |
| 1 | 21.5 | 1.7 | 0.0 |
| 1.5 | 15.2 | 1.3 | 0.3 |
| 2 | 12.4 | 1.9 | 1.8 |
| 2.5 | 11.2 | 3.4 | 11.5 |
| 3 | 9.1 | 3.5 | 50.6 |
| 4 | 5.3 | 20.8 | 91.8 |
| 5 | 2.5 | 8.5 | 27.7 |
| 6 | 1.4 | 3.4 | 13.5 |
| 8 | 0.7 | 0.7 | 4.0 |
| 12 | 0.7 | 0.4 | 1.9 |
| 24 | 0.3 | 0.3 | 0.8 |
| AUC_{0→12 hr} | 72.7 | 46.1 | 204.4 |
| (ng/ml hr) | | | |
| % AUC_{0→12 hr} | 100.0 | 63.4 | 281.2 |
| to C. Ex. 1 | | | |
| Cmax (ng/ml) | 31.8 | 22.1 | 95.3 |
| % Cmax | 100.0 | 69.5 | 299.7 |
| to C. Ex. 1 | | | |

Bisphosphonate-based drugs are known to be absorbed over the gastrointestinal tract [Mitchell et al, Pharm. Res. 15: 228-232 (1998)] and it is reported that the bioavailability of bisphosphonate is impossible to enhance by means of an intestine-targeting drug delivery system, such as enteric coating capsule [Mitchell et al., Pharm. Res. 14: S609 (1996)]. As such, the EDTA-containing enteric coating formulation of Comparative Example 2 was evaluated as not to have increased the bioavailability of bisphosphonate, compared to that of Comparative Example 1.

Despite the report of Mitchell et al., as can be seen from the data of Table 10 and FIG. 5, the phytic acid-containing enteric coating formulation of Example 1, however, increased in both AUC and Cmax by three times, compared to the chelating agent-free commercially available product (Actonel®) of Comparative Example 1 and by four to five times compared to the EDTA-containing enteric coating formulation of Comparative Example 2.

These results were a surprising event because they overturned the prediction of those skilled in the pharmaceutical field that it would be impossible to apply phytic acid to bisphosphonate formulations because an excessive amount thereof would have to be used in order to confer clinically useful bioavailability on them, as naturally deduced from the in vitro tests for inhibition of calcium-risedronate complex formation, showing that the same inhibitory activity against calcium-risedronate complex formation as is obtained by EDTA without using phytic acid in an amount twice that of EDTA.

Based on these results, the present invention provides an oral pharmaceutical formulation that features the enhanced clinical bioavailability of bisphosphonate. Having a low phytic acid content, the oral bisphosphonate formulation of the present invention is unlikely to irritate the intestinal mucosa, with the guaranty of high safety to the patient. Moreover, the oral formulation is designed to allow the patients to take the medicament, together with food intake, at a bioavailability as high as that of an empty stomach, thus improving the convenience of drug administration to the patient. In consequence, the oral formulation of the present invention is expected to provide higher therapeutic effects on osteoporosis, compared to conventional ones.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An oral formulation, comprising bisphosphonate and phytic acid in combination with a delayed release means for releasing the bisphosphonate in a lower gastrointestinal tract.

2. The oral formulation of claim 1, wherein the bisphosphonate is selected from the group consisting of alendronate, risedronate, ibandronate, zoledronate, pamidronate, clodronate, tiludronate, cimadronate and minodronate.

3. The oral formulation of claim 2, wherein the bisphosphonate is risedronate.

4. The oral formulation of claim 1, wherein the delayed release means utilizes an enteric coating drug delivery system capable of releasing the bisphosphonate in a pH-dependent manner.

5. The oral formulation of claim 4, wherein the delayed release means releases the bisphosphonate in a site selected from the group consisting of a duodenum, a jejunum, an ileum and an ascending colon.

6. The oral formulation of claim 4, being designed to slowly release the bisphosphonate during passage through a duodenum, a jejunum, an ileum and an ascending colon.

7. The oral formulation of claim 1, wherein the delayed release means utilizes a delayed release system capable of releasing the bisphosphonate in a time-dependent manner.

8. The oral formulation of claim 7, being designed to release the bisphosphonate within 30 min at a site selected from among a duodenum, a jejunum, an ileum and an ascending colon, during passage therethrough.

9. The oral formulation of claim 7, being designed to release the bisphosphonate at different sites selected from among a duodenum, a jejunum, an ileum and an ascending colon during passage therethrough.

10. The oral formulation of claim 1, wherein the bisphosphonate and the phytic acid are used at a weight ratio of 1:0.5-3.

11. The oral formulation of claim 1, being designed to be administered at a regular time interval selected from among once a day, once a week, once a month, twice a month and three times a month.

12. The oral formulation of claim 11, wherein the regular time interval is once a week.
